(19) United States
(12) Patent Application Publication
(10) Pub. No.: US 2007/0161055 A1
(43) Pub. Date: Jul. 12, 2007

(54) IMMUNOSORBENT ASSAY SUPPORT AND METHOD OF USE

(75) Inventors: Schuyler B. CORRY, Eugene, OR (US); Yue GE, Chapel Hill, NC (US); Iain D. JOHNSON, Eugene, OR (US); Peter SMALLEY, Springfield, OR (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 11/555,127

(22) Filed: Oct. 31, 2006

Related U.S. Application Data
(60) Provisional application No. 60/732,044, filed on Oct. 31, 2005.

(51) Int. Cl. G01N 33/53; C12M 1/34

(52) U.S. Cl. 435/7.5; 435/287.2

(57) ABSTRACT

Embodiments of the present invention provide an immunosorbent assay support immobilized with an intermediate binding antibody and their method of use in an improved immunoassay format.

### CROSS-REFERENCE TO RELATED APPLICATIONS

This application is related to U.S. provisional patent application No. 60/732,044, filed Oct. 31, 2005, from which priority is claimed and which is incorporated by reference in its entirety.

### FIELD OF THE INVENTION

The invention relates to immunosorbent assay supports and to their use in sandwich immunoassays for the detection of a target analyte. The invention has applications in the fields of cell biology, neurology, immunology, pathology and proteomics.

### BACKGROUND OF THE INVENTION

ELISA (Enzyme Linked Immuno-Sorbent Assay) is a widely used and versatile technique that has changed little since its introduction in the 1970's. The underlying technology involves a protein or peptide that is immobilized via passive adsorption on the surface of polystyrene microplate wells. Hydrophobic and charge interactions are responsible for the binding, but not without cost: proteins can denature upon adsorption, which is problematic for antibodies, since the denaturation severely reduces their affinity and binding capacity (Butler J E, et al. (1992) J Immunol Methods 150:77-90). The traditional approach to passively coating antibodies on plates results in a diminution of “active” or “functional” immobilized antibody. Thus, only a portion of the bound antibody is able to capture and subsequently detect the analyte when added to the coated plates.

This problem can be alleviated by immobilizing the capture antibody on the microplate surface via an intermediate coupling interaction. Various coupling interactions have been described including immunospecific interactions (e.g. mouse monoclonal capture antibodies immobilized on microplates coated with goat anti-mouse secondary antibodies), avidin-biotin binding and nucleic acid hybridization (Wacker R, et al. (2004). Anal Biochem. 330:281-287; Vijayendran & Leckband, (2001) Anal Chem. 73:471-480; Peluso et al., (2003). Anal Biochem. 312:113-124; Ross et al., (2000) J Biomed Mater Res. 51:29-36). These methods, while an improvement to passive immobilization also have limitations in that some of the capture antibody may be immobilized in the Fab region, reducing the ability of the capture antibody to bind a target analyte.

Herein we report a new intermediate coupling reaction that increases the amount of active or functional capture antibody that is immobilized on a support and overcomes the limitations of existing methods. This new coupling reaction uses anti-Fc antibodies or anti-Fc antibody fragments that are passively coated on a support and used to immobilize the capture antibody in such a way as to orient them for increased functionality for antigen binding. Using anti-Fc antibodies eliminates the potential of the capture antibody being immobilized by the Fab region. Although the use of Fc-specific secondary antibodies for oriented immobilization of antibodies in affinity chromatography (i.e. purification) has been described (Turkova, (1999) J Chromatogr B Biomed Sci Appl. 722:11-31), their use and advantages in immunoassays (i.e. analyte detection) does not appear to have been previously recognized.

### SUMMARY OF THE INVENTION

Provided in certain embodiments are immunosorbent assay supports that comprise a solid or semi solid support element and an immobilized intermediate binding antibody, where the antibody is typically an anti-Fc antibody or an anti-Fc antibody fragment. The intermediate binding antibody functions to immobilize the capture antibody and thus orienting it away from the support element to increasing the binding of the antibody for the target analyte.

Also provided are methods for detecting a target analyte wherein a sample is added to a present immunosorbent assay support, incubating a support element and sample to form a sample complex, incubating the sample complex with a detection reagent to form a detection complex, illuminating the detection complex and observing the illuminated detection complex to detect the presence or absence of the target analyte.

In another embodiment is provided a kit for the detection of a target analyte comprising an immunosorbent assay support and instructions for using the immunosorbent assay support to detect the target analyte. Addition kit components include buffers, detection reagents and standards.

### BRIEF DESCRIPTION OF THE FIGURES

FIG. 1: Shows the limit of detection determination for Goat anti-Mouse plates from two commercially available sources (BD Biosciences and Pierce Chemical Co.) compared to a present immunosorbent assay support coated with anti-Fc antibody. See Example 3.

FIG. 2: Shows the limit of detection determination for CRP ELISA using either 10 or 100 ng/mL Mouse anti-CRP on Goat anti-Mouse plates from two commercial sources (BD Biosciences and Pierce Chemical Co.) compared to a present immunosorbent assay support coated with anti-Fc antibody. See Example 4.

FIG. 3: Shows the detection of myleoperoxidase (MPO) using a present immunosorbent assay support with goat anti-rabbit IgG HRP as the detection reagent and Amplex UltraRed as the fluorescent substrate. See, Example 5.

FIG. 4: Shows the time dependence for absorption to the wells of a Nunc Maxisorp microplate by a coating antibody, mouse IgG conjugated to Alexa Fluor 555 dye. Error bars represent one standard deviation (12 replicates).

FIG. 5: Shows the concentration dependence of biotin-Mouse IgG binding to wells of a Nunc Maxisorp microplate. Error bars represent one standard deviation (8 replicates).

FIG. 6: Shows the comparison of Mouse anti-biotin activity on an unmodified polystyrene plate versus a goat anti-mouse (GAM) Fc IgG surface. Error bars represent one standard deviation (12 replicates).

### DETAILED DESCRIPTION OF THE INVENTION

Introduction

The present invention provides a superior ELISA support that is able to selectively bind a large quantity of target analyte of interest without denaturation of the capture antibody due to passive absorption. In this instance, anti-Fc antibodies are passively coated on a support element and subsequently used to immobilize the capture antibody in such a way as to orient the Fab region of the capture antibody away from the support element to make it more accessible to the target antigen. In this way the anti-Fc antibody functions as an intermediate binding antibody for the purpose of immobilizing the capture antibody. The use of anti-Fc antibodies also prevents the denaturation of the capture antibody so both the orientation of the Fab region and the lack of denatured capture antibody contribute to the improved antigen detection as compared to standard formats.

Therefore, the use of an immunosorbent assay support coated with Fc-specific intermediate antibodies results in improvements over existing methods in four areas:

1. Shorter incubation time after capture antibody addition (passive adsorption and blocking of capture antibodies generally takes overnight).

2. Capture antibody solutions can be used without purification. Often, mouse IgG is sold in a solution containing BSA or other proteins, often in far greater quantities than the IgG. If these other proteins are not removed before adsorption to polystyrene, they can compete for binding locations on the surface, resulting in even smaller quantities of active mouse IgG on the surface. Because the mode of binding on an anti-mouse Fc plate is immunospecific, as opposed to the non-specific adsorption of a typical ELISA, crude mixtures containing BSA or cell lysate proteins can be used without purification.

3. Smaller amounts of expensive monoclonal capture antibody can be used. This is a result of the preservation of the antigen-binding capacity of the capture antibody resulting from its oriented immobilization by the Fc-specific coating antibody.

4. ELISA signal-to-noise ratios are higher and limits of detection are lower. This is a result of the orientation of the Fab region and the lack of denatured capture antibody.

Definitions

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a ligand” includes a plurality of ligands and reference to “an antibody” includes a plurality of antibodies and the like.

Unless defined otherwise, all technical and scientific terms used herein have the same meaning as commonly understood by one of ordinary skill in the art to which this invention is related. The following terms are defined for purposes of the invention as described herein.

The term “affinity” as used herein refers to the strength of the binding interaction of two molecules, such as an antibody and an antigen or a positively charged moiety and a negatively charged moiety. For bivalent molecules such as antibodies, affinity is typically defined as the binding strength of one binding domain for the antigen, e.g. one Fab fragment for the antigen. The binding strength of both binding domains together for the antigen is referred to as “avidity”. As used herein “High affinity” refers to a ligand that binds to an antibody having an affinity constant (Kₐ) greater than 10⁴ M⁻¹, typically 10⁵-10¹¹ M⁻¹; as determined by inhibition ELISA or an equivalent affinity determined by comparable techniques such as, for example, Scatchard plots or using K_{d}/dissociation constant, which is the reciprocal of the Kₐ, etc.

The term “antibody” as used herein refers to a protein of the immunoglobulin (Ig) superfamily that binds noncovalently to certain substances (e.g. antigens and immunogens) to form an antibody-antigen complex. Antibodies can be endogenous, or polyclonal wherein an animal is immunized to elicit a polyclonal antibody response or by recombinant methods resulting in monoclonal antibodies produced from hybridoma cells or other cell lines. It is understood that the term “antibody” as used herein includes within its scope any of the various classes or sub-classes of immunoglobulin derived from any of the animals conventionally used.

The term “antibody fragments” as used herein refers to fragments of antibodies that retain the principal selective binding characteristics of the whole antibody. Particular fragments are well-known in the art, for example, Fab, Fab′, and F(ab′)₂, which are obtained by digestion with various proteases, pepsin or papain, and which lack the Fc fragment of an intact antibody or the so-called “half-molecule” fragments obtained by reductive cleavage of the disulfide bonds connecting the heavy chain components in the intact antibody. Such fragments also include isolated fragments consisting of the light-chain-variable region, “Fv” fragments consisting of the variable regions of the heavy and light chains, and recombinant single chain polypeptide molecules in which light and heavy variable regions are connected by a peptide linker. Other examples of binding fragments include (i) the Fd fragment, consisting of the VH and CH1 domains; (ii) the dAb fragment (Ward, et al., Nature 341, 544 (1989)), which consists of a VH domain; (iii) isolated CDR regions; and (iv) single-chain Fv molecules (scFv) described above. In addition, arbitrary fragments can be made using recombinant technology that retains antigen-recognition characteristics.

The term “antigen” as used herein refers to a molecule that induces, or is capable of inducing, the formation of an antibody or to which an antibody binds selectively, including but not limited to a biological material. Antigen also refers to “immunogen”. The target-binding antibodies selectively bind an antigen, as such the term can be used herein interchangeably with the term “target”.

The term “anti-region antibody” as used herein refers to an antibody that was produced by immunizing an animal with a select region that is a fragment of a foreign antibody wherein only the fragment is used as the immunogen. Regions of antibodies include the Fc region, hinge region, Fab region, etc. Anti-region antibodies include monoclonal and polyclonal antibodies. The term “anti-region fragment” as used herein refers to a monovalent fragment that was generated from an anti-region antibody of the present invention by enzymatic cleavage.

The term “aqueous solution” as used herein refers to a solution that is predominantly water and retains the solution characteristics of water. Where the aqueous solution contains solvents in addition to water, water is typically the predominant solvent.

The term “buffer” as used herein refers to a system that acts to minimize the change in acidity or basicity of the solution against addition or depletion of chemical substances.

The term “capture antibody” as used herein refers to a an antibody that has specificity for a target analyte. In this instance, the capture antibody is not passively coated on a support but immobilized by the use of an intermediate antibody, such as anti-Fc antibody.

The term “chromophore” as used herein refers to a label that emits light in the visible spectra that can be observed without the aid of instrumentation.

The term “complex” as used herein refers to the association of two or more molecules, usually by non-covalent bonding, e.g., the association between an antibody and an antigen or the labeling reagent and the target-binding antibody.

The term “detectable response” as used herein refers to an occurrence of, or a change in, a signal that is directly or indirectly detectable either by observation or by instrumentation.

Typically, the detectable response is an occurrence of a signal wherein the fluorophore is inherently fluorescent and does not produce a change in signal upon binding to a metal ion or biological compound. Alternatively, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density.

The term “detectably distinct” as used herein refers to a signal that is distinguishable or separable by a physical property either by observation or by instrumentation. For example, a fluorophore is readily distinguishable either by spectral characteristics or by fluorescence intensity, lifetime, polarization or photo-bleaching rate from another fluorophore in the sample, as well as from additional materials that are optionally present.

The term “directly detectable” as used herein refers to the presence of a material or the signal generated from the material is immediately detectable by observation, instrumentation, or film without requiring chemical modifications or additional substances.

The term “fluorophore” as used herein refers to a composition that is inherently fluorescent or demonstrates a change in fluorescence upon binding to a biological compound or metal ion, i.e., fluorogenic. Fluorophores may contain substitutents that alter the solubility, spectral properties or physical properties of the fluorophore. Numerous fluorophores are known to those skilled in the art and include, but are not limited to coumarin, cyanine, benzofuran, a quinoline, a quinazolinone, an indole, a benzazole, a borapolyazaindacene and xanthenes including fluoroscein, rhodamine and rhodol as well as other fluorophores described in RICHARD P. HAUGLAND, MOLECULAR PROBES HANDBOOK OF FLUORESCENT PROBES AND RESEARCH CHEMICALS (gth edition, CD-ROM, September 2002).

The term “intermediate binding antibody” as used herein refers to an antibody that is passively coated on a surface but does not have affinity for the target analyte. Instead the intermediate binding antibody has affinity for the capture or primary antibody. The intermediate binding antibody is also an unlabeled secondary antibody.

The term “kit” as used herein refers to a packaged set of related components, typically one or more compounds or compositions.

The term “label” as used herein refers to a chemical moiety or protein that retains it's native properties (e.g. spectral properties, conformation and activity) when attached to a labeling reagent and used in the present methods. The label can be directly detectable (fluorophore) or indirectly detectable (hapten or enzyme). Such labels include, but are not limited to, radiolabels that can be measured with radiation-counting devices; pigments, dyes or other chromogens that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems, for example. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a calorimetric substrate (e.g., tetramethylbenzidine (TMB)) or a fluorogenic substrate such as Amplex Red reagent (Molecular Probes, Inc.) to detect the presence of HRP. Numerous labels are know by those of skill in the art and include, but are not limited to, particles, fluorophores, haptens, enzymes and their calorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9^{th} edition, CD-ROM, September 2002), supra.

The terms “protein” and “polypeptide” are used herein in a generic sense to include polymers of amino acid residues of any length. The term “peptide” is used herein to refer to polypeptides having less than 100 amino acid residues, typically less than 10 amino acid residues. The terms apply to amino acid polymers in which one or more amino acid residues are an artificial chemical analogue of a corresponding naturally occurring amino acid, as well as to naturally occurring amino acid polymers.

The term “purified” as used herein refers to a preparation of a target-binding antibody that is essentially free from contaminating proteins that normally would be present in association with the antibody, e.g., in a cellular mixture or milieu in which the protein or complex is found endogenously such as serum proteins or hybridoma supernatant.

The term “sample” as used herein refers to any material that may contain an analyte for detection or quantification. The analyte may include a reactive group, e.g., a group through which a compound of the invention can be conjugated to the analyte. The sample may also include diluents, buffers, detergents, and contaminating species, debris and the like that are found mixed with the target. Illustrative examples include urine, sera, blood plasma, total blood, saliva, tear fluid, cerebrospinal fluid, secretory fluids from nipples and the like. Also included are solid, gel or sol substances such as mucus, body tissues, cells and the like suspended or dissolved in liquid materials such as buffers, extractants, solvents and the like. Typically, the sample is a live cell, a biological fluid that comprises endogenous host cell proteins, nucleic acid polymers, nucleotides, oligonucleotides, peptides and buffer solutions. The sample may be in an aqueous solution, a viable cell culture or immobilized on a solid or semi solid surface such as a polyacrylamide gel, membrane blot or on a microarray.

The term “support element” refers to an adsorbent solid or semi-solid support for immobilizing anti-Fc antibodies, which includes include a bead, a particle, an array, a glass slide or a multiwell plate. Columns, such as affinity columns, which are used for purification and not detection of analytes, specifically those described in Turkova, (1999). J Chromatogr B Biomed Sci Appl. 722:11-31; are not support elements of the present invention.

The term “target” as used herein refers to any entity that a target-binding antibody has affinity for such as an epitope or antigen. This target includes not only the discrete epitope that the target-binding antibody has affinity for but also includes any subsequently bound molecules or structures. In this way an epitope serves as a marker for the intended target. For example, a cell is a target wherein the target-binding antibody binds a cell surface protein such as CD3 on a T cell wherein the target marker is CD3 and the target is the T cell.

The term “target-binding antibody” as used herein refers to an antibody that has affinity for a discrete epitope or antigen that can be used with the methods of the present invention. Typically the discrete epitope is the target but the epitope can be a marker for the target such as CD3 on T cells. The term can be used interchangeably with the term “primary antibody” or “capture antibody” when describing methods that use an antibody that binds directly to the antigen as opposed to a “secondary antibody” that binds to a region of the primary antibody.

The Immunosorbent Assay Support

In general, for ease of understanding the present invention, the immunosorbent assay support will first be described in detail, followed by the many and varied methods in which the immobilized anti-Fc antibody or fragment thereof find uses, which is followed by exemplified methods.

Provided is an immunosorbent assay support comprising a solid or semi solid support element that is passively absorbed with immobilized intermediate binding antibodies. In a typical immunoassay, which consist of a coating of monoclonal “capture” antibody, followed by the sample to be measured, then a polyclonal “detection” antibody with affinity for the same protein as the monoclonal, the capture antibody is typically denatured to a degree that reducing the antibodies antigen. In the present invention the intermediate binding antibody is passively immobilized on a support element wherein the intermediate binding antibody has affinity for a capture antibody, thus eliminating the need to passively absorb the capture antibody and reducing and/or eliminating the denaturing effects on the capture antibody.

In addition to maintaining the integrity of the capture antibody, the present immunosorbent assay support further enhances the availability of the capture antibody binding sites for the target analyte by orienting them away from the support element. This is accomplished by using an intermediate binding antibody that has selective affinity for the Fc region of the capture antibody. In this way the maximum distance between the support element and the binding site of the capture antibody is achieved. As is demonstrated in the Examples section, this configuration of support element, intermediate binding antibody and capture antibody resulted in a surprising increasing in detection of an analyte as seen in the detection limit, dynamic range and concentration of intermediate antibody and capture antibody required per assay. The present immunosorbent assay support demonstrates a significant step forward in immunosorbent assay technology resulting in an improved immunosorbent assay system that has seen little change in the last 30 years.

The intermediate binding antibody is any polyclonal or monoclonal antibody that has affinity for the Fc region of a capture antibody. As used herein, a “functional fragment” of an immunoglobulin is a portion of the immunoglobulin molecule that specifically binds to a binding target. The intermediate binding antibody also includes Fab, Fab′ or F(ab′)₂ that have affinity for the Fc region of the capture antibody wherein the intermediate binding antibody may be a mixture of intact antibodies and fragments or a homogenous mixture of fragments or interact antibodies.

The intermediate binding antibodies of the present invention may also be described or specified in terms of their cross-reactivity, as well as their binding affinity towards the antigen. Specific examples of binding affinities encompassed in the present invention include but are not limited to those with a dissociation constant (Kd) less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Antibody is a term of the art denoting the soluble substance or molecule secreted or produced by an animal in response to an antigen, and which has the particular property of combining specifically with the antigen that induced its formation. Antibodies themselves also serve are antigens or immunogens because they are glycoproteins and therefore are used to generate anti-species antibodies, such as an anti-goat Fc antibody. Antibodies, also known as immunoglobulins, are classified into five distinct classes—IgG, IgA, IgM, IgD, and IgE. The basic IgG immunoglobulin structure consists of two identical light polypeptide chains and two identical heavy polypeptide chains (linked together by disulfide bonds). These chains can be cleaved to form fragments (anti-Fc fragments) or an Fc fragment to be used as an immunogen to generate the anti-Fc antibody. As used herein, the term antibody is used to mean immunoglobulin molecules and functional fragments thereof, regardless of the source or method of producing the fragment. Whole antibodies may be monoclonal or polyclonal, and they may be humanized or chimeric. The term “monoclonal antibody” as used herein is not limited to antibodies produced through hybridoma technology. Rather the term “monoclonal antibody” refers to an antibody that is derived from a single clone, including any eukaryotic, prokaryotic, or phage clone, and not the method by which it is produced.

The antibodies of the present invention may be monospecific, bispecific, trispecific or of even greater multispecificity. In addition the antibodies may be monovalent, bivalent, trivalent or of even greater multivalency. Furthermore, the antibodies of the invention may be from any animal origin including, but not limited to, birds and mammals. In specific embodiments, the antibodies are human, murine, rat, sheep, rabbit, goat, guinea pig, horse, or chicken. In an exemplary embodiment, the intermediate binding antibodies of the present invention are produced from either murine monoclonal antibodies or polyclonal antibodies generated in a variety of animals that have been immunized with a foreign antibody or fragment thereof, U.S. Pat. No. 4,196,265 discloses a method of producing monoclonal antibodies. Typically, intermediate binding antibodies are derived from a polyclonal antibody that has been produced in a rabbit or goat but any animal known to one skilled in the art to produce polyclonal antibodies can be used to generate anti-species antibodies. However, monoclonal antibodies are equal, and in some cases, preferred over polyclonal antibodies provided that the capture antibody is compatible with the monoclonal antibodies that are typically produced from murine hybridoma cell lines using methods well known to one skilled in the art. Example 1 and 2 of US 20030073149 (those examples are herein incorporated by reference) describes production of polyclonal antibodies raised in animals immunized with the Fc region of a foreign antibody. It is a preferred embodiment of the present invention that the intermediate binding antibody be generated against only the Fc region of a foreign antibody. Essentially, the animal is immunized with only the Fc region fragment of a foreign antibody, such as murine. The polyclonal antibodies are collected from subsequent bleeds to produce the intermediate binding antibodies. The intermediate binding antibodies are then affinity purified on a column comprising Fc fragments that the animal was immunized against. In addition, many commercial suppliers exist for anti-Fc antibodies, including Immunology Consulting Laboratories, See Example 1 below.

The intermediate binding antibodies are passively absorbed on a solid or semi-solid support element using methods well known in the art. The support element, includes any immunoassay-based support system wherein passive absorption of the intermediate antibody is possible and wherein the supports facilitate an immunosorbent assay. Well known supports include a bead, a particle, an array, a glass slide or a multiwell plate. The supports of the present invention are not columns for use in purification based methods involving affinity chromatography. The multiwell plates are particularly advantageous for multiple sample analysis and the plates are available commercially from a number of suppliers, in a number of compositions and formats. In an exemplary embodiment polystyrene multiwell plates are used.

Thus, provided in one embodiment is a method for preparing an immunosorbent assay support, comprising the steps:
i. providing a support that is a solid or semi solid support;
ii. contacting the support with an aqueous solution comprising anti-Fc antibodies or anti-Fc antibody fragments;
iii. incubating the support and the aqueous solution for a sufficient amount of time to allow the anti-Fc antibodies or an anti-Fc antibody fragments to become immobilized to form an immobilized support; and,
iv. removing the aqueous solution from the immobilized support wherein an immunosorbent assay support is prepared.

A solid support suitable for use in the present invention is typically substantially insoluble in liquid phases. A large number of supports are available and are known to one of ordinary skill in the art. Useful solid supports include solid and semi-solid matrixes, such as aerogels and hydrogels, beads, biochips (including thin film coated biochips), microfluidic chip, a silicon chip, multi-well plates (also referred to as microtitre plates or microplates), membranes, conducting and nonconducting metals, glass (including microscope slides) and magnetic supports. More specific examples of useful solid supports include polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

In one embodiment is provided an immunosorbent assay support for detection of a target analyte, where the immunosorbent assay support comprises an antibody immobilized on a support; wherein;
a) the support is a solid or semi-solid support;
b) the antibody is an anti-Fc antibody or an anti-Fc antibody fragment that functions to immobilize an analyte capture antibody that is used to bind and subsequently detect a target analyte in an immunosorbent assay.

The intermediate binding antibody is passively absorbed on the support using techniques well known in the art, See Example 2. There is no intended limitation on the method including the buffers and incubation times used to immobilize the intermediate binding antibody on the support. Many standard protocols call for an overnight incubation for the coating antibody, however we have found that this length of time is likely unnecessary, See Example 6.

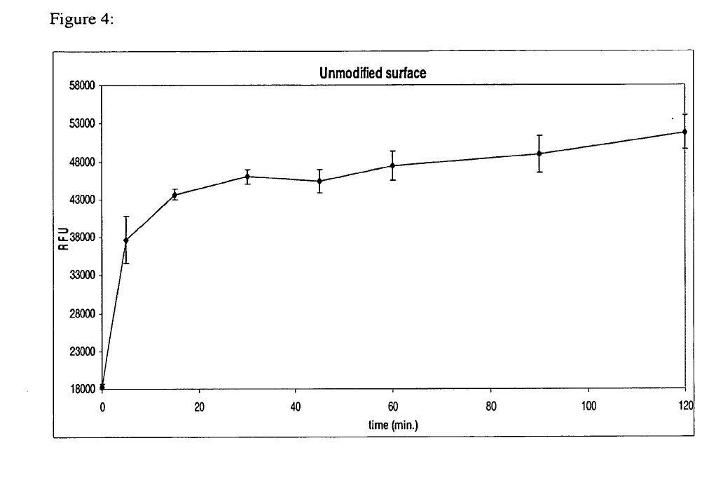

To test the hypothesis of length of time necessary for absorption of coating antibody, plates were coated with mouse IgG labeled with Alexa Fluor 555 Dye. This particular antibody was chosen because the dye is very bright, resistant to quenching, and has minimal nonspecific binding interactions. This experiment, as seen in FIG. 4, demonstrates that initial binding is very rapid. After five minutes, the signal is about 58% of its final value; after fifteen minutes, it has reached 76% of the final value (background subtracted for calculations). It is also apparent that the signal is low compared to the background, in spite of the brightness of the dye; even the brightest signal is not even three times above noise. However, the dim signal is good enough to indicate that typical overnight incubation times are excessive, and 1-2 hours incubation is sufficient. Thus, the range of incubation times for immobilization of the intermediate binding antibody range from as short as five minutes to as long as overnight, with the optimal incubation time being at least one hour but not more than six hours. However, due to workflow constraints it is often advantageous to incubate the coating antibody overnight.

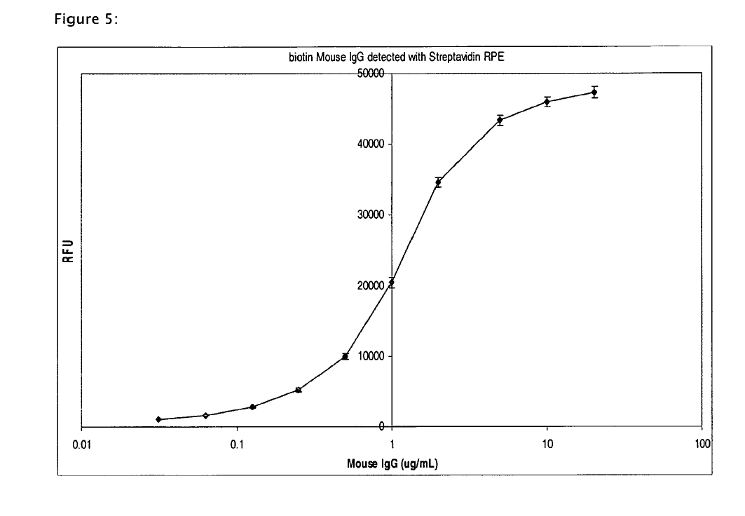

To further analyze the binding capacity of multiwell plates an experiment was designed to determine the saturation point of coating antibody on the plates. To overcome the limitations of Alexa Fluor 555 Mouse IgG, a different detection scheme was devised, employing RPE as the fluorescent reporter. Besides having a very high quantum yield, RPE has a very long Stokes shift (495 nm/575 nm ex/em) that significantly reduces background fluorescence. See Example 7 and FIG. 5. As IgG concentration increases, the quantity of bound protein asymptotically reaches saturation, meaning an infinite concentration of IgG is required to fully saturate the surface. Since this is impossible, the half-saturation point, which is also the point of inflection, is a more realistic way to measure saturation. The point of inflection can be mathematically determined by curve-fitting; in the FIG. 5, the half-saturation concentration of Mouse IgG is 1.2 μg/mL. In practice, however, it is not the total quantity of bound antibody that is important, but the quantity of bound antibody that retains its binding capacity. Thus, it is important to determine the amount of bound antibody that remains active to demonstrate the significant improvement the use of the present intermediate binding antibody plays in an immunosorbent assay.

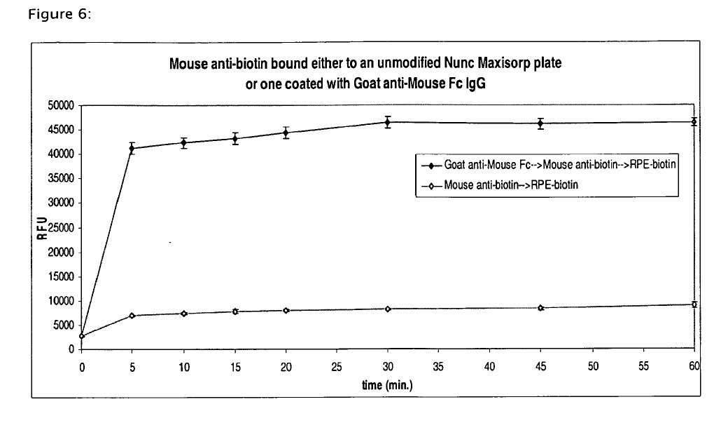

As with the incubation time and plate (polystyrene) capacity experiments, a system to quantify antibody binding capacity should have as few amplification steps as possible. An antibody to a fluorescent reagent would be ideal, as long as the fluorescence were not adversely affected by binding. This rules out the most obvious monoclonal candidate, mouse anti-fluorescein, because the fluorescence is quenched upon binding. Mouse anti-biotin and several biotin-dye conjugates are available, including Alexa Fluor 555 dye. An approach using biotinylated RPE was used, again because of its high quantum yield and long Stokes shift. Mouse anti-biotin was applied to a microplate, followed by the RPE-biotin reagent, and the absolute quantity of RPE was calculated using a standard curve. Example 8 demonstrates the difference in signal obtained when plates are coated with biotinylated IgG and detected with RPE-Streptavidin (SA) and those coated with an intermediate binding antibody anti-Fc IgG, See FIG. 6. Thus, demonstrating the improvement of using the intermediate binding antibody to retain functionality of the capture antibody in an immunoassay.

This was further tested by comparing to commercially available plates that are immobilized with anti-species antibodies, but not specific for the Fc region. BD Falcon and Pierce both sell Goat anti-Mouse IgG precoated microplates, but neither manufacturer indicates that the coating antibody is specific to the Fc region. Although the BD and Pierce plates both have high capacities for Mouse IgG, the manner of immobilization of Mouse IgG is probably more heterogeneous than for the Goat anti-Mouse IgG Fc plates.

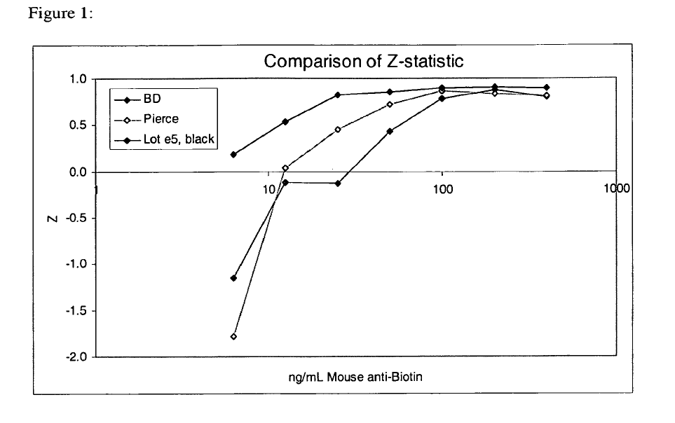

To test this hypothesis, an experiment was designed to determine how little Mouse IgG is necessary to distinguish signal from noise. To determine the limit of detection of Mouse IgG, a dilution series of Mouse anti-Biotin (400-6.25 ng/mL) was applied to the BD, Pierce, and the present anti-Fc IgG plates, followed by a fixed concentration of RPE-SS-Biotin (1000 ng/mL), and the limit of detection calculated by the Z-statistic, See FIG. 1 and Example 1. The Z-statistic, and therefore signal-to-noise, is clearly higher for the anti-Fc IgG plates than for those made by BD and Pierce. Additionally, the total amount of RPE-SS-Biotin captured was higher for the anti-Fc IgG plates than for the BD and Pierce plates for all concentrations of Mouse anti-Biotin except the highest (400 ng/mL). This confirms that although the anti-Fc IgG plates have lower capacity for Mouse IgG, any IgG that is captured is more active (indicated by the total RPE captured) and less heterogeneous (indicated by the Z-statistic and error bars).

In summary, this disclosure concerns the use of Fc-specific secondary antibodies as intermediary coatings between microplate surfaces and capture antibodies. Exemplary embodiments include, microplates coated with goat anti-mouse Fc antibody and then blocked with BSA, using concentrations and buffers well known in the art. For use in a sandwich ELISA, the coated plate is incubated with capture antibody for about 30 minutes, but there is no intended limitation on the incubation time, after which experimental samples can be added immediately. Experimentally we have determined that much smaller amounts of capture antibody (e.g. 10 ng/mL) can be used compared to assays run using microplates coated with non-Fc-specific secondary antibodies (typically requiring 100 ng/mL of capture antibody). The amount of functional capture antibody deposited can be assessed using an anti-biotin antibody in combination with biotinylated R-phycoerythrin, see Example 8.

Thus, in one embodiment is provided an immunosorbent assay support wherein the amount of active capture antibody is at least 10% more than a normal passively absorbed capture antibody. In one aspect the immunosorbent assay support comprising an immobilized anti-Fc antibody, wherein the anti-Fc antibody immobilizes capture antibody and orients the capture antibody to increase the percentage of immobilized capture antibody that are active for binding a target analyte.

Method of Use

The present invention also provides methods of using the compounds described herein to detect an analyte in a sample. Those of skill in the art will appreciate that this focus is for clarity of illustration and does not limit the scope of the methods in which the compounds of the invention find use.

Provided in one embodiment in a method for detecting a target analyte, wherein the method comprises:
a) contacting an immunosorbent assay support with a primary antibody that is selective for a target analyte to form a primary antibody complexed support, wherein the immunosorbent assay support comprises a solid or semi-solid support that is immobilized with anti-Fc antibody or anti-Fc antibody fragment;
b) contacting the primary antibody complexed support with a sample to form a sample complex;
c) contacting the sample complex with a detection reagent to form a detection complex;
d) illuminating the detection complex to form an illuminated sample; and, observing the illuminated sample to detect the presence or absence of the target analyte.

Another embodiment provides a method for the detection of a target analyte in an immunosorbent assay, wherein the method comprises:
providing a support that is a solid or semi solid support;
contacting the support with an aqueous solution comprising anti-Fc antibodies or anti-Fc antibody fragments;
incubating the support and the aqueous solution for a sufficient amount of time to allow the anti-Fc antibodies or an anti-Fc antibody fragments to become immobilized to form an immobilized support; and,
removing the aqueous solution from the immobilized support wherein an immunosorbent assay support is prepared;
contacting the immunosorbent assay support with a primary antibody that is selective for a target analyte to form a primary antibody complexed support;
contacting the primary antibody complexed support with a sample to form a sample complex;
contacting the sample complex with a detection reagent to form detection complex;
illuminating the detection complex to form an illuminated sample; and,
observing the illuminated sample to detect the presence or absence of the target analyte.

Another embodiment provides method for the detection of a target analyte, wherein the method comprises:
contacting an immunosorbent assay support with a sample comprising a target analyte to form a sample complex, wherein the immunosorbent assay support comprises a support element immobilized with anti-Fc antibody or anti-Fc antibody fragment immobilized to a capture antibody; and
detecting binding of the target analyte to the capture antibody.

The detection reagent can be any label or reporter molecule conjugated to a specific binding partner. Typically this would be an antibody, antigen, biotin or streptavidin, all conjugates typically used in an immunoassay. However, there is no intended limitation of the specific binding partner that can be conjugated to a label and used in the present methods to detect a target analyte.

TABLE 2
Representative Specific Binding Pairs

| antigen | antibody |
|---|---|
| biotin | avidin (or streptavidin or anti-biotin) |
| IgG* | protein A or protein G |
| drug | drug receptor |
| folate | folate binding protein |
| toxin | toxin receptor |
| carbohydrate | lectin or carbohydrate receptor |
| peptide | peptide receptor |
| protein | protein receptor |
| enzyme substrate | enzyme |
| Fc region | Anti-Fc antibody |
| hormone | hormone receptor |
| ion | chelator |

The labels of the present invention include any directly or indirectly detectable label known by one skilled in the art that can be covalently attached to a specific binding partner. Labels include, without limitation, a chromophore, a fluorophore, a fluorescent protein, a phosphorescent dye, a tandem dye, a particle, a hapten, an enzyme and a radioisotope. Preferred labels include fluorophores, fluorescent proteins, haptens, and enzymes.

A fluorophore of the present invention is any chemical moiety that exhibits an absorption maximum beyond 280 nm, and when covalently attached to a labeling reagent retains its spectral properties. Fluorophores of the present invention include, without limitation; a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Ser. Nos. 09/968,401 and 09/969,853), a carbocyanine (including any corresponding compounds in U.S. Ser. Nos. 09/557,275; 09/969,853 and 09/968,401; U.S.; Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; and publications WO 02/26891, WO 97/40104, WO 99/51702, WO 01/21624; EP 1 065 250 A1), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and U.S. Ser. No. 09/922,333), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

When the fluorophore is a xanthene, the fluorophore is optionally a fluorescein, a rhodol (including any corresponding compounds disclosed in U.S. Pat. Nos. 5,227,487 and 5,442,045), or a rhodamine (including any corresponding compounds in U.S. Pat. Nos. 5,798,276; 5,846,737; U.S. Ser. No. 09/129,015). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171). Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position.

Preferred fluorophores of the invention include xanthene (rhodol, rhodamine, fluorescein and derivatives thereof) coumarin, cyanine, pyrene, oxazine and borapolyazaindacene. Most preferred are sulfonated xanthenes, fluorinated xanthenes, sulfonated coumarins, fluorinated coumarins and sulfonated cyanines. The choice of the fluorophore attached to the labeling reagent will determine the absorption and fluorescence emission properties of the labeling reagent and immuno-labeled complex. Physical properties of a fluorophore label include spectral characteristics (absorption, emission and stokes shift), fluorescence intensity, lifetime, polarization and photo-bleaching rate all of which can be used to distinguish one fluorophore from another.

Typically the fluorophore contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

In one aspect of the invention, the fluorophore has an absorption maximum beyond 480 nm. In a particularly useful embodiment, the fluorophore absorbs at or near 488 nm to 514 nm (particularly suitable for excitation by the output of the argon-ion laser excitation source) or near 546 nm (particularly suitable for excitation by a mercury arc lamp).

Many of fluorophores can also function as chromophores and thus the described fluorophores are also preferred chromophores of the present invention.

In addition to fluorophores, enzymes also find use as labels for the detection reagents. Enzymes are desirable labels because amplification of the detectable signal can be obtained resulting in increased assay sensitivity. The enzyme itself does not produce a detectable response but functions to break down a substrate when it is contacted by an appropriate substrate such that the converted substrate produces a fluorescent, calorimetric or luminescent signal. Enzymes amplify the detectable signal because one enzyme on a labeling reagent can result in multiple substrates being converted to a detectable signal. This is advantageous where there is a low quantity of target present in the sample or a fluorophore does not exist that will give comparable or stronger signal than the enzyme. However, fluorophores are most preferred because they do not require additional assay steps and thus reduce the overall time required to complete an assay. The enzyme substrate is selected to yield the preferred measurable product, e.g. calorimetric, fluorescent or chemiluminescence. Such substrates are extensively used in the art, many of which are described in the MOLECULAR PROBES HANDBOOK, supra.

A preferred calorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other calorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including Amplex® Red reagent and its variants (U.S. Pat. No. 4,384,042), Amplex UltraRed and its variants in (WO05042504) and reduced dihydroxanthenes, including dihydrofluoresceins (U.S. Pat. No. 6,162,931) and dihydrorhodamines including dihydrorhodamine 123. Peroxidase substrates that are tyramides (U.S. Pat. Nos. 5,196,306; 5,583,001 and 5,731,158) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

Another preferred colorimetric (and in some cases fluorogenic) substrate and enzyme combination uses a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912) fluorescein diphosphate, 3-O-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates (U.S. Pat. Nos. 5,316,906 and 5,443,986).

Glycosidases, in particular beta-galactosidase, beta-glucuronidase and beta-glucosidase, are additional suitable enzymes. Appropriate colorimetric substrates include, but are not limited to, 5-bromo-4-chloro-3-indolyl beta-D-galactopyranoside (X-gal) and similar indolyl galactosides, glucosides, and glucuronides, o-nitrophenyl beta-D-galactopyranoside (ONPG) and p-nitrophenyl beta-D-galactopyranoside. Preferred fluorogenic substrates include resorufin beta-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236), 4-methylumbelliferyl beta-D-galactopyranoside, carboxyumbelliferyl beta-D-galactopyranoside and fluorinated coumarin beta-D-galactopyranosides (U.S. Pat. No. 5,830,912).

Additional enzymes include, but are not limited to, hydrolases such as cholinesterases and peptidases, oxidases such as glucose oxidase and cytochrome oxidases, and reductases for which suitable substrates are known.

Enzymes and their appropriate substrates that produce chemiluminescence are preferred for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In addition to enzymes, haptens such as biotin are also preferred labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

Fluorescent proteins also find use as labels for the labeling reagents of the present invention. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. For this to work, the fluorescent protein and fluorophore function as an energy transfer pair wherein the fluorescent protein emits at the wavelength that the fluorophore absorbs at and the fluorophore then emits at a wavelength farther from the fluorescent proteins than could have been obtained with only the fluorescent protein. A particularly useful combination is the phycobiliproteins disclosed in U.S. Pat. Nos. 4,520,110; 4,859,582; 5,055,556 and the sulforhodamine fluorophores disclosed in 5,798,276, or the sulfonated cyanine fluorophores disclosed in U.S. Ser. Nos. 09/968/401 and 09/969/853; or the sulfonated xanthene derivatives disclosed in U.S. Pat. No. 6,130,101 and those combinations disclosed in U.S. Pat. No. 4,542,104. Alternatively, the fluorophore functions as the energy donor and the fluorescent protein is the energy acceptor.

Sample Preparation

The end user will determine the choice of the sample and the way in which the sample is prepared. The sample includes, without limitation, any biological derived material or aqueous solution that is believed to contain a target analyte or ligand. Alternatively, samples also include material in which an analyte or ligand has been added.

The sample can be a biological fluid such as whole blood, plasma, serum, nasal secretions, sputum, saliva, urine, sweat, transdermal exudates, cerebrospinal fluid, or the like. Biological fluids also include tissue and cell culture medium wherein an analyte of interest has been secreted into the medium. Alternatively, the sample may be whole organs, tissue or cells from the animal. Examples of sources of such samples include muscle, eye, skin, gonads, lymph nodes, heart, brain, lung, liver, kidney, spleen, thymus, pancreas, solid tumors, macrophages, mammary glands, mesothelium, and the like. Cells include without limitation prokaryotic cells and eukaryotic cells that include primary cultures and immortalized cell lines. Eukaryotic cells include without limitation ovary cells, epithelial cells, circulating immune cells, β cells, hepatocytes, and neurons.

One may use an individual compound of the invention, multiple compounds of the invention or a combination of a compound of the invention and a fluorophore or quencher of a different structure in order to detect the presence of or determine the characteristics of a target in a sample.

When the components of the invention are species that bind to targets that are specific biological structures (e.g., enzymes, receptors, ligands, antigens, antibodies, etc.), the reaction time between the compound or conjugate of the invention and the target will usually be at least about 5 min, more usually at least about 30 min and preferably not more than about 180 min, preferably not more than about 120 min, depending upon the temperature, concentrations of enzyme and substrate, etc. By using a specific time period for the reaction or taking aliquots at 2 different times, the rate of reaction can be determined for comparison with other determinations. The temperature will generally be in the range of about 20 to 50° C., more usually in the range of about 25 to 40° C.

Various buffers can be used in the assays of the invention. These buffers include PBS, Tris, MOPS, HEPES, phosphate, etc. The pH will vary depending upon the particular assay system, generally within a readily determinable range wherein one or more of the sulfonic acid moieties is deprotonated. The concentration of buffer is generally in the range of about 0.1 to 50 mM, more usually 0.5 to 20 mM.

In many instances, it may be advantageous to add a small amount of a non-ionic detergent to the sample. Generally the detergent will be present in from about 0.01 to 0.1 vol. %. Illustrative non-ionic detergents include the polyoxyalkylene diols, e.g. Pluronics, Tweens, Triton X-100, etc.

In fluorescence experiments, the reaction is optionally quenched. Various quenching agents may be used, both physical and chemical. Conveniently, a small amount of a water-soluble solvent may be added, such as acetonitrile, DMSO, SDS, methanol, DMF, etc.

Illumination

The compounds of the invention may, at any time after or during an assay, be illuminated with a wavelength of light that results in a detectable optical response, and observed with a means for detecting the optical response. Upon illumination, such as by an violet or visible wavelength emission lamp, an arc lamp, a laser, or even sunlight or ordinary room light, the fluorescent compounds, including those bound to the complementary specific binding pair member, display intense visible absorption as well as fluorescence emission. Selected equipment that is useful for illuminating the fluorescent compounds of the invention includes, but is not limited to, hand-held ultraviolet lamps, mercury arc lamps, xenon lamps, argon lasers, laser diodes, and YAG lasers. These illumination sources are optionally integrated into laser scanners, flow cytometer, fluorescence microplate readers, standard or mini fluorometers, or chromatographic detectors. This fluorescence emission is optionally detected by visual inspection, or by use of any of the following devices: CCD cameras, video cameras, photographic film, laser scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, a fluorescence microscope or a fluorometer, the instrument is optionally used to distinguish and discriminate between the fluorescent compounds of the invention and a second fluorophore with detectably different optical properties, typically by distinguishing the fluorescence response of the fluorescent compounds of the invention from that of the second fluorophore. Where a sample is examined using a flow cytometer, examination of the sample optionally includes isolation of particles within the sample based on the fluorescence response by using a sorting device.

Kits of the Invention

In another aspect, the present invention provides kits that include an immunosorabent assay support of the invention. The kit will generally also include instructions for using the support of the invention in one or more methods, typically for the detection of a target analyte.

In an exemplary embodiment, the kit includes an immunosorbent assay support, wherein the immunosorbent assay support comprises a solid or semi-solid support that is immobilized with anti-Fc antibodies or anti-Fc antibody fragments; and instructions to use the immunosorbent assay support for detection of a target analyte in an immunosorbent assay. Additional kit components include buffers, detection reagents and standards.

Particular Aspects of the Invention:

One aspect of the invention provides an immunosorbent assay support comprising an anti-Fc antibody immobilized on a support element.

Another aspect of the invention provides a method for the detection of a target analyte, wherein the method comprises:
contacting an immunosorbant assay support with a primary antibody that is selective for a target analyte to form a primary antibody complexed support element, wherein the immunosorbant assay support comprises a support element that is immobilized with anti-Fc antibody or anti-Fc antibody fragment;
contacting the primary antibody complexed support element with a sample to form a sample complex;
contacting the sample complex with a detection reagent to form a detection complex;
illuminating the detection complex to form an illuminated sample; and,
observing the illuminated sample to detect the presence or absence of the target analyte.

Another aspect of the invention provides a kit for the detection of a target analyte, wherein the kit comprises;
an immunosorbent assay support, wherein the immunosorbent assay support comprises a support element that is immobilized with anti-Fc antibodies or anti-Fc antibody fragments; and
instructions to use the immunosorbent assay support for detection of a target analyte in an immunosorbent assay.

More particularly, the capture antibody is immobilized on the anti-Fc antibody. More particular still, the capture antibody binds a target analyte and the capture antibody is oriented to increase binding of the target analyte. More particularly, the capture antibody is an anti-biotin antibody. More particular still, the target analyte is biotinylated.

In another embodiment, the support element is solid or the support element is semi-solid. In another embodiment the support element is a bead, a particle, an array, a glass slide or a multiwell plate. More particularly, the support element is a bead.

Another embodiment comprises a plurality of different capture antibodies.

In another embodiment, the immunosorbent assay support is substantially free of impurities. More particularly, the impurities comprise bovine serum albumin (BSA).

In another embodiment, the anti-Fc antibody is a mouse antibody.

In another embodiment, a target analyte bound to the capture antibody. More particularly, a secondary antibody bound to the target analyte. More particularly, a label bound to the support element, anti-Fc antibody, target analyte, capture antibody or a secondary capture antibody that binds the target analyte. More particular still, the label is a fluorophore or an enzyme.

In an additional aspect of any of the foregoing embodiment, the antibody, particularly the anti-Fc antibody is an antibody fragment. More particular still the fragment is a Fab, Fab′, and F(ab′)₂.

Another aspect of the invention provides an immunosorbant assay support wherein the amount of active capture antibody is at least 10% more than a normal passively absorbed capture antibody. More particularly, the aspect further comprises an immobilized anti-Fc antibody. More particularly, the anti-Fc antibody immobilizes capture antibody and orients the capture antibody to increase the percentage of immobilized capture antibody that are active for binding a target analyte.

Another aspect of the invention provides an immunosorbant assay support for detection of a target analyte, where the immunosorbant assay support comprises an antibody immobilized on a support; wherein;
the support is a solid or semi-solid support;
the antibody is an anti-Fc antibody or an anti-Fc antibody fragment that functions to immobilize an analyte capture antibody that is used to bind and subsequently detect a target analyte in an immunosorbent assay.

In another embodiment, the solid or semi-solid support is a bead, a particle, an array, a glass slide or a multiwell plate.

A detailed description of the invention having been provided above, the following examples are given for the purpose of illustrating the invention and shall not be construed as being a limitation on the scope of the invention or claims.

A detailed description of the invention having been provided above, the following examples are given for the purpose of illustrating the invention and shall not be construed as being a limitation on the scope of the invention or claims.

### EXAMPLES

### Example 1

### Preparation of Microplates Coated with Goat Anti-Mouse IgG₁ Fc Antibody

10 mg of cross-adsorbed goat anti-Mouse IgG₁ Fc (Immunology Consulting Laboratories) were added to 1 L of 100 mM sodium bicarbonate/phosphate buffered saline (PBS), pH 9.3 to make a 10 μg/mL solution. 100 μL of this solution was dispensed into the wells of a 96-well microplate with high protein binding capacity (Greiner BioOne, catalog number 655081). After incubation for 4-8 hours, the antibody solution was removed from the plates and replaced by 1% (10 g/L) bovine serum albumin (BSA) blocking solution (200 μL/well). After incubation overnight at room temperature in the dark, the blocking solution was removed and the plates were placed face down on blotting paper, protected from light, until dry. Plates were stored, protected from light, at room temperature or at 4° C. until required for use.

### Example 2

### Preparation of R-PE-SS-biotin

2 mL of 20 mg/mL R-phycoerythrin (R-PE) suspension in 60% saturated (NH₄)₂SO₄, 50 mM KPᵢ, pH 7.0 was centrifuged for 15 minutes at 10,000 rpm. After removal of the supernatant, the protein pellet was dissolved in 2 mL of 0.1 M sodium phosphate, 0.1 M NaCl, pH 7.5. This solution was then dialyzed against 4 changes of 2 L of 0.1 M sodium phosphate, 0.1 M NaCl, pH 7.5. The R-PE concentration of this solution was determined by absorption spectrophotometry (A₅₆₅ nm=8.17 for 1 mg/mL R-PE) to be 12 mg/mL. 0.25 mL (3 mg) of dialyzed R-PE was transferred to a reaction vessel, to which 25 μL of 1 M sodium bicarbonate (pH 9.0) was then added. 9.1 μL of a 10 mg/mL solution of sulfo-NHS—SS-biotin (Pierce Chemical Co., catalog number 21331) in DMSO was added dropwise to the stirred R-PE solution in the reaction vessel. The reaction vessel was covered and the reaction mixture was stirred for 1 hour at ambient temperature. The R-PE-SS-biotin conjugate was purified by column chromatography on a 1×20 cm column of Bio-Gel P-30 (Bio-Rad Laboratories) eluted with 0.1 M sodium phosphate, 0.1 M NaCl, pH 7.5 and the final concentration of the product was determined by absorption spectrophotometry (A₅₆₅ nm).

### Example 3

### Determination of Limits of Detection for Mouse Monoclonal Anti-Biotin Antibody

96-well polystyrene microplates coated with 1 μg/well of goat anti-mouse IgG₁ Fc antibody were prepared as described in example 1. Microplates coated with goat anti-mouse IgG (H+L) antibodies were obtained from Pierce Chemical (catalog number 15134) and BD Biosciences (catalog number 354170). Samples of mouse anti-biotin IgG₁ antibody (monoclonal 2F5; Molecular Probes, Inc. A11242) with a concentration range of 6.25-400 ng/mL were applied to all three microplates, followed by 1 μg/mL R-PE-SS-biotin (Example 2). All wells were washed three times with phosphate buffered saline (PBS) and then treated with 50 mM dithiothreitol in PBS, resulting in release of R-PE into solution in proportion to the amount of biotin captured by the anti-biotin antibody. R-PE fluorescence intensities for each sample were measured in a fluorescence microplate reader using excitation at 490 nm and emission detection at 570 nm. The results are expressed as Z statistic (Zhang et al., 1999) versus anti-biotin concentration. The higher the Z value at any particular analyte concentration, the better is the ability of the assay to detect that concentration of analyte above the analyte-free background. The results demonstrate the capacity of microplates coated with Fc-specific anti-mouse antibodies to enable the use of smaller quantities of valuable capture antibodies in sandwich ELISAs without detriment to the signal-to-noise characteristics of the assay. See, FIG. 1.

### Example 4

### Comparison of Limits of Detection for C-Reactive Protein (CRP) ELISA

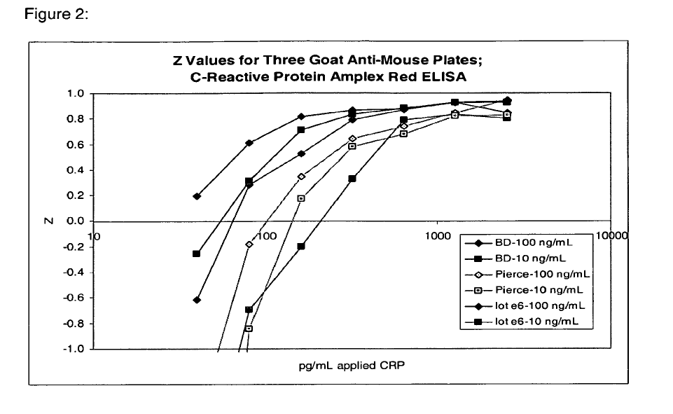

96-well polystyrene microplates coated with 1 μg/well of goat anti-mouse IgG₁ Fc antibody were prepared as described in example 1. Microplates coated with goat anti-mouse IgG (H+L) antibodies were obtained from Pierce Chemical (catalog number 15134) and BD Biosciences (catalog number 354170). Mouse monoclonal anti-CRP capture antibody (Fitzgerald Industries catalog number 10-C33, clone number M701289) was added to each plate at 100 ng/mL (48/96 wells) or 10 ng/mL (48/96 wells). Samples of CRP-reactive protein varying in concentration from 40 μg/mL to 2560 μg/mL were added. After 60 minutes incubation, all wells were washed three times with phosphate buffered saline (PBS)-Tween. Goat anti-CRP horseradish peroxidase conjugate (Rockland Immunochemicals; 500 ng/mL), H₂O₂ (200 μM) and Amplex Red fluorogenic peroxidase substrate (50 μM) were added to all samples. After incubation for 60 minutes, the fluorescence intensity of each sample was measured in a fluorescence microplate reader using excitation at 530 nm and emission detection at 590 nm. The results are expressed as Z statistic (Zhang et al., 1999) versus CRP concentration. The higher the Z value at any particular analyte concentration, the better is the ability of the assay to detect that concentration of analyte above the analyte-free background. The results show that microplates coated with goat anti-mouse IgG₁ Fc antibody provide lower limits of detection in the CRP ELISA than microplates coated with goat anti-mouse IgG (H+L) antibodies. See, FIG. 2.

### Example 5

### Detection of Myeloperoxidase (MPO) Using a Goat Anti-Mouse IgG₁ Fc Antibody Coated Plate

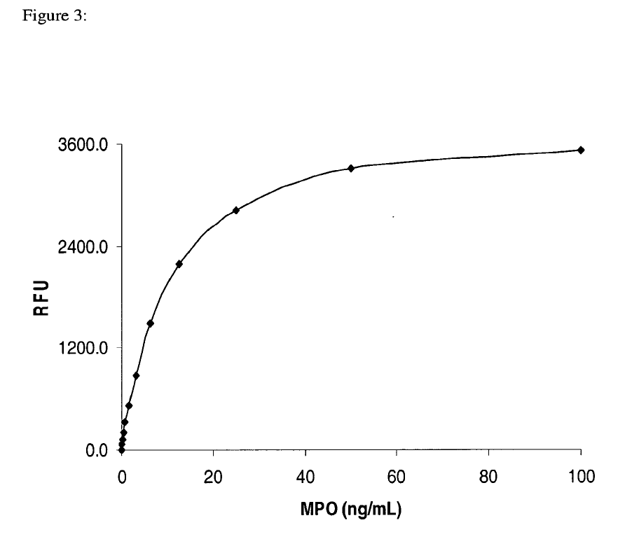

96-well polystyrene microplates coated with 1 μg/well of goat anti-mouse IgG₁ Fc antibody were prepared as described in example 1. 100 μL of anti-myeloperoxidase monoclonal antibody (BD Bioscience) at the concentration of 500 ng/mL was added into each well of the anti-Fc antibody coated plate. The plate was incubated on a plate shaker for 1 hr at ˜500 rpm at room temperature, the contents of the wells emptied and washed three times with 200 μL of 1×PBS-Teen. After the final wash, the wells were emptied or aspirated. 100 μL of serially diluted myeloperoxidase standard was then added into each well. Each concentration of sample was triplicated. Briefly, myeloperoxidase (US Biological) was diluted to 100 ng/mL using 0.1×PBS-BSA. The solution was then serially diluted two fold using the same buffer. 100 μl of the preparations were added in each well of the anti-Fc antibody coated plate. 100 μL of anti-MPO polyclonal antibody (Invitrogen) at the concentration of 330 ng/mL was added into each well and incubated for 30 minutes. After washing 100 μL of goat anti-rabbit IgG HRP (Invitrogen) solution at the concentration of 100 ng/mL was added to each well and incubated for 30 minutes. After washing, 100 μL of Amplex UltraRed (Invitrogen) and H₂O₂ (Invitrogen) mixture was added into each well. The mixture was made by adding 50 μL of 10 mM Amplex UltraRed and 22.7 μL of 3% H₂O₂ to 10 mL of 1×PBS. The plate was incubated at room temperature or 37° C. for 30 min protected from light. The fluorescence absorbance was read in a PerSeptive Biosystems CytoFluor 4000 microplate plate reader with an 530 nm excitation filter and a 580 nm emission filter, with a gain setting of 35. The dynamic range of the assay is between 0.2 ng/mL and 100 ng/mL. To the best of our knowledge this is the first time a fluorescent ELISA assay for myeloperoxidase has been presented. See, FIG. 3.

### Example 6

### Plate Binding Assay

100 μL of mouse IgG labeled with Alexa Fluor 555 dye, with a degree of labeling (DOL) of 5.2 dyes per antibody, diluted to 10 μg/mL in a pH 9.3 solution of 100 mM bicarbonate mixed with phosphate buffered saline (PBS), were added successively to rows of a Nunc Maxisorp microplate at various time points. After two hours and seven time points, the plate was then drained and washed three times with PBS/0.05% Tween-20. Results are presented in FIG. 4.

### Example 7

### Plate Binding Capacity

A dilution series of biotinylated Mouse IgG in a PBS/bicarbonate buffer was applied to a microplate, allowed to incubate overnight (to eliminate the time variable), then drained and blocked for 90 minutes with 1% BSA in PBS. After washing, the biotin-Mouse IgG was detected with 10 μg/mL streptavidin-RPE. Results are shown in FIG. 5.

### Example 8

### Comparison of passively coated anti-biotin IgG and anti-Fc IgG

Plates were coated with anti-Fc IgG as described in Example 1. Plates were coated with 10 μg/mL Mouse anti-biotin. For plates coated with anti-Fc IgG, the anti-biotin antibody was added and then subsequently detected with RPE-biotin, in which the RPE moiety is bound to biotin through a disulfide bond (i.e. RPE-SS-biotin). See FIG. 6.

The reagents employed in the examples are commercially available or can be prepared using commercially available instrumentation, methods, or reagents known in the art. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

We claim:

1. An immunosorbent assay support comprising an anti-Fc antibody immobilized on a support element.

2. The immunosorbent assay support according to claim 1, comprising a capture antibody immobilized on the anti-Fc antibody.

3. The immunosorbent assay support according to claim 2, wherein the capture antibody binds a target analyte and the capture antibody is oriented to increase binding of the target analyte.

4. The immunosorbent assay support according to claim 1, wherein the support element is solid.

5. The immunosorbent assay support according to claim 1, wherein the support element is semi-solid.

6. The immunosorbent assay support according to claim 1, wherein the support element is a bead, a particle, an array, a glass slide or a multiwell plate.

7. The immunosorbent assay support according to claim 3, wherein the capture antibody is an anti-biotin antibody.

8. The immunosorbent assay support according to claim 7, wherein the target analyte is biotinylated.

9. The immunosorbent assay support according to claim 2, comprising a plurality of different capture antibodies.

10. The immunosorbent assay support according to claim 9, wherein the support element is a bead.

11. The immunosorbent assay support according to claim 1, substantially free of impurities.

12. The immunosorbent assay support according to claim 11, wherein the impurities comprise bovine serum albumin (BSA).

13. The immunosorbent assay support according to claim 2, wherein the anti-Fc antibody is a mouse antibody.

14. The immunosorbent assay support according to claim 2, comprising a target analyte bound to the capture antibody.

15. The immunosorbent assay support according to claim 14, comprising a secondary antibody bound to the target analyte.

16. The immunosorbent assay support according to claim 14, comprising a label bound to the support element, the anti-Fc antibody, the target analyte, the capture antibody or bound to a secondary capture antibody that binds the target analyte.

17. The immunosorbent assay support according to claim 15, wherein the label is a fluorophore or an enzyme.

18. A method for the detection of a target analyte, wherein the method comprises:
a) contacting an immunosorbant assay support with a primary antibody that is selective for a target analyte to form a primary antibody complexed support element, wherein the immunosorbant assay support comprises a support element that is immobilized with anti-Fc antibody or anti-Fc antibody fragment;
b) contacting the primary antibody complexed support element with a sample to form a sample complex;
c) contacting the sample complex with a detection reagent to form a detection complex;
d) illuminating the detection complex to form an illuminated sample; and,
e) observing the illuminated sample to detect the presence or absence of the target analyte.

19. A kit for the detection of a target analyte, wherein the kit comprises;
a) an immunosorbent assay support, wherein the immunosorbent assay support comprises a support element that is immobilized with anti-Fc antibodies or anti-Fc antibody fragments; and
b) instructions to use the immunosorbent assay support for detection of a target analyte in an immunosorbent assay.
